(19) United States
(12) Patent Application Publication
(10) Pub. No.: US 2006/0240032 A1
(43) Pub. Date: Oct. 26, 2006

(54) Immunomodulating compositions and methods for use in the treatment of human autoimmune diseases

(76) Inventors: David J. Hinrichs, Lake Oswego, OR (US); Dan V. Mourich, Albany, OR (US); Patrick L. Iversen, Corvallis, OR (US)

Correspondence Address: PERKINS COIE LLP, P.O. BOX 2168, MENLO PARK, CA, 94026, US

(21) Appl. No.: 11/395,663

(22) Filed: Mar. 30, 2006

Related U.S. Application Data
(60) Provisional application No. 60/667,236, filed on Mar. 31, 2005.

(51) Int. Cl. A61K 39/00

(52) U.S. Cl. 424/185.1

(57) ABSTRACT

A composition and method for treating an autoimmune condition are disclosed. The composition includes of an immunomodulating compound composed of a central amino acid core having a plurality of chemical attachment groups, and a plurality of antigenic peptides which are (i) associated with an auto-immune disorder and (ii) attached to the core groups with the same N-terminus to C-terminus orientation. The compound is carried in a pharmaceutically acceptable carrier. An exemplary compound has an octomeric polylysine core and eight antigenic peptides, such as the peptides identified by SEQ ID NOS: 1-11, attached thereto. The compound is effective in treating an autoimmune disorder, by administering to a subject in need of the treatment, a pharmaceutically effective amount of the compound.

This patent application claims priority to U.S. provisional patent application No. 60/667,236 filed on Mar. 31, 2005, which is incorporated in its entirety herein by reference.

### FIELD OF THE INVENTION

The present invention relates to novel drug compounds and methods to regulate the immune response by the inhibition or destruction of an antigen-responsive cell at the time of its response to antigen. Specifically, the compounds and methods of the invention can be used to treat human autoimmune diseases including Type I diabetes, multiple sclerosis, rheumatoid arthritis, psoriasis, pemphigus vulgaris and Celiac disease.

### REFERENCES

Amagai, M., V. Klaus-Kovtun, et al. (1991). “Autoantibodies against a novel epithelial cadherin in pemphigus vulgaris, a disease of cell adhesion.” Cell 67(5): 869-77.

Bielekova, B., B. Goodwin, et al. (2000). “Encephalitogenic potential of the myelin basic protein peptide (amino acids 83-99) in multiple sclerosis: results of a phase II clinical trial with an altered peptide ligand.” Nat Med 6(10): 1167-75.

Bielekova, B., M. H. Sung, et al. (2004). “Expansion and functional relevance of high-avidity myelin-specific CD4+T cells in multiple sclerosis.” J Immunol 172(6): 3893-904.

Bourdette, D. N., A. A. Vandenbark, et al. (1989). “T cell lines selected with synthetic peptides are highly encephalitogenic in SJL/J mice.” J Neuroimmunol 22(3): 255-60.

Bracci, L., C. Falciani, et al. (2003). “Synthetic peptides in the form of dendrimers become resistant to protease activity.” J Biol Chem 278(47): 46590-5.

Burrows, G. G., K. L. Adlard, et al. (2000). “Regulation of encephalitogenic T cells with recombinant TCR ligands.” J Immunol 164(12): 6366-71.

Burrows, G. G., B. F. Bebo, Jr., et al. (1998). “Two-domain MHC class II molecules form stable complexes with myelin basic protein 69-89 peptide that detect and inhibit rat encephalitogenic T cells and treat experimental autoimmune encephalomyelitis.” J Immunol 161(11): 5987-96.

Chao, C. C. and H. 0. McDevitt (1997). “Identification of immunogenic epitopes of GAD 65 presented by Ag7 in non-obese diabetic mice.” Immunogenetics 46(1): 29-34.

Chou, Y. K., I. Robey, et al. (1998). “Induction of T cell anergy by high concentrations of immunodominant native peptide is accompanied by IL-10 production and a block in JNK activity.” Cell Immunol 188(2): 125-36.

Cross, A. H., V. K. Tuohy, et al. (1993). “Development of reactivity to new myelin antigens during chronic relapsing autoimmune demyelination.” Cell Immunol 146(2): 261-9.

Daniel, D. and D. R. Wegmann (1996). “Protection of nonobese diabetic mice from diabetes by intranasal or subcutaneous administration of insulin peptide B-(9-23).” Proc Natl Acad Sci USA 93(2): 956-60.

de Rosbo, N. K., J. F. Kaye, et al. (2004). “The myelin-associated oligodendrocytic basic protein region MOBP15-36 encompasses the immunodominant major encephalitogenic epitope(s) for SJL/J mice and predicted epitope(s) for multiple sclerosis-associated HLA-DRB1*1501.” J Immunol 173(2): 1426-35.

Ellis, H. J., E. L. Pollock, et al. (2003). “Investigation of the putative immunodominant T cell epitopes in coeliac disease.” Gut 52(2): 212-7.

Ellis, T. M. and M. A. Atkinson (1996). “The clinical significance of an autoimmune response against glutamic acid decarboxylase.” Nat Med 2(2): 148-53.

Falk, K., O. Rotzschke, et al. (2000). “Induction and suppression of an autoimmune disease by oligomerized T cell epitopes: enhanced in vivo potency of encephalitogenic peptides.” J Exp Med 191(4): 717-30.

Falk, K., O. Rotzschke, et al. (2000). “Antigen-specific elimination of T cells induced by oligomerized hemagglutinin (HA) 306-318.” Eur J Immunol 30(10): 3012-20.

Fraser, J. S., W. Engel, et al. (2003). “Coeliac disease: in vivo toxicity of the putative immunodominant epitope.” Gut 52(12): 1698-702.

Fritz, R. B., C. H. Chou, et al. (1983). “Relapsing murine experimental allergic encephalomyelitis induced by myelin basic protein.” J Immunol 130(3): 1024-6.

Fujii, S., K. Liu, et al. (2004). “The linkage of innate to adaptive immunity via maturing dendritic cells in vivo requires CD40 ligation in addition to antigen presentation and CD80/86 costimulation.” J Exp Med 199(12): 1607-18.

Greer, J. M., P. A. Csurhes, et al. (2004). “Effect of gender on T-cell proliferative responses to myelin proteolipid protein antigens in patients with multiple sclerosis and controls.” J Autoimmun 22(4): 345-52.

Harrison, L. C. and D. A. Hafler (2000). “Antigen-specific therapy for autoimmune disease.” Curr Opin Immunol 12(6): 704-11.

Hawiger, D., K. Inaba, et al. (2001). “Dendritic cells induce peripheral T cell unresponsiveness under steady state conditions in vivo.” J Exp Med 194(6): 769-79.

Holz, A., B. Bielekova, et al. (2000). “Myelin-associated oligodendrocytic basic protein: identification of an encephalitogenic epitope and association with multiple sclerosis.” J Immunol 164(2): 1103-9.

Huan, J., S. Subramanian, et al. (2004). “Monomeric recombinant TCR ligand reduces relapse rate and severity of experimental autoimmune encephalomyelitis in SJL/J mice through cytokine switch.” J Immunol 172(7): 4556-66.

Jones, D. A., N. Yawalkar, et al. (2004). “Identification of autoantigens in psoriatic plaques using expression cloning.” J Invest Dermatol 123(1): 93-100.

Kappos, L., G. Comi, et al. (2000). “Induction of a non-encephalitogenic type 2 T helper-cell autoimmune response in multiple sclerosis after administration of an altered peptide ligand in a placebo-controlled, randomized phase II trial. The Altered Peptide Ligand in Relapsing MS Study Group.” Nat Med 6(10): 1176-82.

Kent, S. C., Y. Chen, et al. (2005). “Expanded T cells from pancreatic lymph nodes of type 1 diabetic subjects recognize an insulin epitope.” Nature 435(7039): 224-8.

Larsen, C. E. and C. A. Alper (2004). “The genetics of HLA-associated disease.” Curr Opin Immunol 16(5): 660-7.

Lin, M. S., S. J. Swartz, et al. (1997). “Development and characterization of desmoglein-3 specific T cells from patients with pemphigus vulgaris.” J Clin Invest 99(1): 31-40.

Liu, E., H. Moriyama, et al. (2002). “Anti-peptide autoantibodies and fatal anaphylaxis in NOD mice in response to insulin self-peptides B:9-23 and B: 13-23.” J Clin Invest 110(7): 1021-7.

Liu, E., H. Moriyama, et al. (2004). “Preventing peptide-induced anaphylaxis: addition of C-terminal amino acids to produce a neutral isoelectric point.” J Allergy Clin Immunol 114(3): 607-13.

Lu, Y. A., P. Clavijo, et al. (1991). “Chemically unambiguous peptide immunogen: preparation, orientation and antigenicity of purified peptide conjugated to the multiple antigen peptide system.” Mol Immunol 28(6): 623-30.

Mannie, M. D., S. K. Rendall, et al. (1996). “Anergy-associated T cell antigen presentation. A mechanism of infectious tolerance in experimental autoimmune encephalomyelitis.” J Immunol 157(3): 1062-70.

Mantegazza, R., P. Cristaldini, et al. (2004). “Anti-MOG autoantibodies in Italian multiple sclerosis patients: specificity, sensitivity and clinical association.” Int Immunol 16(4): 559-65.

Markovic-Plese, S., C. Pinilla, et al. (2004). “The initiation of the autoimmune response in multiple sclerosis.” Clin Neurol Neurosurg 106(3): 218-22.

Nakayama, M., N. Abiru, et al. (2005). “Prime role for an insulin epitope in the development of type 1 diabetes in NOD mice.” Nature 435(7039): 220-3.

Pender, M. P. and N. P. Wolfe (2002). “Prevention of autoimmune attack and disease progression in multiple sclerosis: current therapies and future prospects.” Intern Med J 32(11): 554-63.

Rotzschke, O., K. Falk, et al. (1997). “Superactivation of an immune response triggered by oligomerized T cell epitopes.” Proc Natl Acad Sci USA 94(26): 14642-7.

Shan, L., O. Molberg, et al. (2002). “Structural basis for gluten intolerance in celiac sprue.” Science 297(5590): 2275-9.

Steinman, L. (2004). “Immune therapy for autoimmune diseases.” Science 305(5681): 212-6.

Stienekemeier, M., K. Falk, et al. (2001). “Vaccination, prevention, and treatment of experimental autoimmune neuritis (EAN) by an oligomerized T cell epitope.” Proc Natl Acad Sci USA 98(24): 13872-7.

Tam, J. P. (1996). “Recent advances in multiple antigen peptides.” J Immunol Methods 196(1): 17-32.

Tuohy, V. K., Z. Lu, et al. (1989). “Identification of an encephalitogenic determinant of myelin proteolipid protein for SJL mice.” J Immunol 142(5): 1523-7.

Tuohy, V. K., D. M. Thomas, et al. (1995). “Determinant-regulated onset of experimental autoimmune encephalomyelitis: distinct epitopes of myelin proteolipid protein mediate either acute or delayed disease in SJL/J mice.” Autoimmunity 21(3): 203-14.

van Gaalen, F. A., J. van Aken, et al. (2004). “Association between HLA class II genes and autoantibodies to cyclic citrullinated peptides (CCPs) influences the severity of rheumatoid arthritis.” Arthritis Rheum 50(7): 2113-21.

Vandenbark, A. A., B. Celnik, et al. (1995). “Myelin antigen-coupled splenocytes suppress experimental autoimmune encephalomyelitis in Lewis rats through a partially reversible anergy mechanism.” J Immunol 155(12): 5861-7.

Vandenbark, A. A., C. Rich, et al. (2003). “Recombinant TCR ligand induces tolerance to myelin oligodendrocyte glycoprotein 35-55 peptide and reverses clinical and histological signs of chronic experimental autoimmune encephalomyelitis in HLA-DR2 transgenic mice.” J Immunol 171(1): 127-33.

Vanderlugt, C. L., K. L. Neville, et al. (2000). “Pathologic role and temporal appearance of newly emerging autoepitopes in relapsing experimental autoimmune encephalomyelitis.” J Immunol 164(2): 670-8.

Veldman, C., A. Stauber, et al. (2003). “Dichotomy of autoreactive Thl and Th2 cell responses to desmoglein 3 in patients with pemphigus vulgaris (PV) and healthy carriers of PV-associated HLA class II alleles.” J Immunol 170(1): 635-42.

Veldman, C. M., K. L. Gebhard, et al. (2004). “T cell recognition of desmoglein 3 peptides in patients with pemphigus vulgaris and healthy individuals.” J Immunol 172(6): 3883-92.

von Herrath, M. (2005). “Immunology: insulin trigger for diabetes.” Nature 435(7039): 151-2.

Whitham, R. H., R. E. Jones, et al. (1991). “Location of a new encephalitogenic epitope (residues 43 to 64) in proteolipid protein that induces relapsing experimental autoimmune encephalomyelitis in PL/J and (SJL×PL)F1 mice.” J Immunol 147(11): 3803-8.

You, S., C. Chen, et al. (2004). “Presence of diabetes-inhibiting, glutamic acid decarboxylase-specific, IL-10-dependent, regulatory T cells in naive nonobese diabetic mice.” J Immunol 173(11): 6777-85.

Yu, M., J. M. Johnson, et al. (1996). “A predictable sequential determinant spreading cascade invariably accompanies progression of experimental autoimmune encephalomyelitis: a basis for peptide-specific therapy after onset of clinical disease.” J Exp Med 183(4): 1777-88.

Zamvil, S., P. Nelson, et al. (1985). “T-cell clones specific for myelin basic protein induce chronic relapsing paralysis and demyelination.” Nature 317(6035): 355-8.

### BACKGROUND OF THE INVENTION

The immune response is required to combat infectious disease and to function as an internal surveillance mechanism for the elimination of cells that express alterations due to mutation. In many clinical settings, however, control or complete inhibition of the immune response is desired. This is most evident in allogeneic organ transplantation but is also required in order to combat autoimmune disease, as well as the side effects of immune responses as evident in allergies and some forms of asthma. Since currently used immunosuppression strategies are not selective, the induced level of immunosuppression and its duration of administration must be balanced by the need for the immune response to be able to respond to endogenous and exogenous stimuli as required. An ideal immunosuppression strategy would allow selective elimination of the unwarranted or detrimental immune response while retaining the beneficial immune response potential as needed. None of the clinical strategies currently in use can achieve such selection.

For autoimmune disease, different strategies to achieve immunosuppression are required in that the clinical appearance of the autoimmune disease is a function of the effector stage of the immune response. Thus, strategies must be developed that target not only this effector arm responsible for the ongoing autoimmune episode, but they must also have an impact on the immunologic pathway that has promoted the cellular events to develop to a point that the autoimmune disease is evident. If successful at the effector T cell level, such strategies would presumably have an immediate effect on clinical disease. Since chronic as well as relapsing autoimmune diseases are likely to require the continued development and differentiation of the pathogenic immune response, strategies designed to inhibit this development would have long term rather than an immediate influence on clinical disease.

Regulation of the immune response is needed in many clinical settings. However, virtually no clinically approved drugs to inhibit the immune response are selective and their use entails significant side effects. The development of a strategy to selectively inhibit or destroy an antigen-responsive cell at the time of its response to antigen would be of significant benefit and allow for the development of aggressive approaches to the regulation of the immune response. For T cell mediated autoimmune disease, both the effector T-cell and its development and differentiation are dependent on the recognition of and response to a cognate antigen. Consequently, many immunosuppressive strategies employ the presumed autoantigen in various forms in order to achieve the goal of specifically inhibiting the expression as well as the development of the autoimmune response. The goal of specifically eliminating the effector activity of the immune response, preventing the continued development of this pathogenic lymphoid pathway and maintaining the potential for all other immunologic responses is widely recognized as an unmet medical need. Modification of the immune response using multimers of the epitope in a well-defined animal model provide the guidance needed to apply this strategy to human autoimmune disease. Therefore, the present invention addresses the unmet medical need to develop a general approach for antigen specific intervention in autoimmune disease.

### SUMMARY OF THE INVENTION

In one aspect, the invention includes a composition for use in treating an autoimmune condition. The composition includes an immunomodulating compound composed of a central amino acid core having a plurality of chemical attachment groups, and a plurality of antigenic peptides which are (i) associated with an auto-immune disorder and (ii) attached to said groups with the same N-terminus to C-terminus orientation. The compound is carried in a pharmaceutically acceptable carrier.

Each of the antigenic peptides may optionally be attached to the amino acid core through a spacer containing a linear chain of at least about 10 atoms in length. Exemplary chains include a string of at least 5 amino acids, such as the spacer having SEQ ID NO:12. In another embodiment, the antigenic peptides may optionally be attached to the amino acid core through a spacer containing a linear chain of at least about 5 atoms in length, where an exemplary chain includes a string of at least 2 amino acids. The central amino acid core is a branched polylysine, which may have 4-16 attachment sites, with the compound having between 4-16 antigenic peptides. An exemplary core is composed of a polylysine octamer, with the compound including up to eight antigenic peptides, each coupled to the octamer through the peptide's C terminus.

Exemplary peptide antigens include those identified by SEQ ID NOS:1-11. The carrier may be sterile buffered physiological saline or other liquid suitable for injection into a human subject. Favorable routes of injection include, for example, subcutaneous and intravenous injection.

In another aspect, the invention includes a method of treating an autoimmune disorder in a mammalian subject in need of such treatment by administering to the subject, in a pharmaceutically effective amount, a composition containing an immunomodulating antigen-specific compound composed of a central amino acid core having a plurality of chemical attachment groups, and a plurality of antigenic peptides which are (i) associated with an auto-immune disorder and (ii) attached to said groups with the same N-terminus to C-terminus orientation. The compound is carried in a pharmaceutically acceptable carrier.

For use in treating multiple sclerosis, the compound administered may contain, as the antigenic peptides, a peptide selected from the group consisting of SEQ ID NOS: 1, 7, or 8.

For use in treating early-onset diabetes, the compound administered may contain, as the antigenic peptides, a peptide selected from the group consisting of SEQ ID NOS:2, 9, 10,or 11.

For use in treating Pemphigus vulgaris, the compound administered may contain, as the antigenic peptides, a peptide selected from the group consisting of SEQ ID NOS:3-5.

For use in treating Celiac disease, the compound administered may contain, as the antigenic peptides, the peptide identified as SEQ ID NO:6.

These and other objects and features of the present invention will become more fully apparent when the following detailed description of the invention is read in conjunction with the accompanying drawings.

### BRIEF DESCRIPTION OF THE DRAWINGS

FIG. 1. Structure of the MAP139 octamer showing eight PLP₍₁₃₉₋₁₅₁₎ peptides (NH₃-HSLGKWLGHPDKF—COOH, SEQ ID NO:8) linked through a spacer (NH₃—KKKKKGG-COOH, SEQ ID NO: 11) to a central lysine-branched core molecule.

FIG. 2. EAE induced in SJL mice by PLP₍₁₃₉₋₁₅₁₎ immunization and treatment with MAP139 octamer. SJL mice were immunized with the PLP₍₁₃₉₋₁₅₁₎ peptide in CFA. Groups of mice were treated 2, 6 and 10 days following immunization.

FIG. 3. Dose titration of EAE disease inhibition by the MAP139 octamer. SJL mice were immunized with the PLP₍₁₃₉₋₁₅₁₎ peptide in CFA. Groups of mice were treated 2, 6 and 10 days following immunization.

FIG. 4. Relapsing EAE induced by immunization with PLP₍₁₃₉₋₁₅₁₎. MAP139 octamer effectively inhibits rEAE. SJL mice were immunized with the PLP₍₁₃₉₋₁₅₁₎ peptide in CFA. Treatment was initiated after the development of clinical EAE.

FIG. 5. Treatment with MAP139 octamer does not inhibit the development of PLP₍₁₇₈₋₁₉₁₎ induced rEAE. SJL mice were immunized with the PLP₍₁₇₈₋₁₉₁₎ peptide in CFA. On day 2, 6 and 10 following immunization mice were injected with a saline solution of the MAP139 octamer.

FIG. 6. EAE induced by the transfer of PLP₍₁₃₉₋₁₅₁₎ specific T-cells and treatment with the MAP139 octamer inhibits the development of rEAE. All recipients were injected with 10 million PLP₍₁₃₁₋₁₅₁₎ peptide-specific T-cells. Treatment was initiated after signs of clinical disease were apparent.

### DETAILED DESCRIPTION OF THE INVENTION

### DEFINITIONS

An antigenic peptide is “associated with an autoimmune disorder” if the peptide has the potential to provoke an autoimmune response-that is, a response to self-in a subject, e.g., a human subject. Examples of such peptides are those identified with SEQ ID NOS:1-10 below.

Antigenic peptides are “attached to attachment groups with the same N-terminus to C-terminus orientation” if each peptide is attached to its associated attachment group at its C-terminus, in one embodiment, or at its N-terminus, in another embodiment.

The terms “administration” or “administering” refer to a method of incorporating a compound into the cells or tissues of a subject, either in vivo or ex vivo to diagnose, prevent, treat, or ameliorate a symptom of a disease. In one example, a compound can be administered to a subject in vivo parenterally. In another example, a compound can be administered to a subject by combining the compound with cell tissue from the subject ex vivo for purposes that include, but are not limited to, cell expansion and mobilization assays. When the compound is incorporated in the subject in combination with one or active agents, the termis “administration” or “administering” can include sequential or concurrent incorporation of the compound with the other agents such as, for example, any agent described above. A pharmaceutical composition of the invention is formulated to be compatible with its intended route of administration. Examples of routes of administration include, but are not limited to, for example, intravenous, intradermal, intramuscular, subcutaneous injection; oral; inhalation; vaginal; aural; nasal; intranasal; transdermal; transmucosal; pulmonary; intravenous; intracranial; intraperitoneal; subcutaneous; intramuscular and rectal administration.

An “effective amount” of a compound of the invention can be used to describe a therapeutically effective amount or a prophylactically effective amount. A “therapeutically effective amount” refers to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect. In some embodiments, the therapeutically effective amount may need to be administered in an amount sufficient to result in amelioration of one or more symptoms of a disorder, prevention of the advancement of a disorder, or regression of a disorder. In one example, treatment of an inflammatory disorder or an autoimmune disorder characterized by inflammation, a therapeutically effective amount preferably refers to the amount of a therapeutic agent that reduces the inflammation of a joint, organ or tissue by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. The term “treating” refers to the administering one or more diagnostic, therapeutic or prophylactic agents.

I. Human Autoimmune Diseases

Autoimmune diseases are the result of the immune system producing a cellular and/or humoral immune response that reacts with autoantigens. While such an autoimmune response is a relatively infrequent occurrence within the general population, its development is of great concern clinically with many autoimmune diseases contributing to long-term disease and death. The events that initiate human autoimmune disease are not defined, although for most autoimmune diseases a genetically defined clustering is evident, especially regarding the Major Histocompatibility Complex, (MHC) (Larsen and Alper 2004; van Gaalen, van Aken et al. 2004). Examples of such MHC associations are evident for Type I diabetes (Larsen and Alper 2004), Multiple Sclerosis (Markovic-Plese, Pinilla et al. 2004), rheumatoid arthritis (van Gaalen, van Aken et al. 2004), psoriasis (Jones, Yawalkar et al. 2004) and the inflammatory bowel disease (celiac disease) associated with gluten intolerance (Shan, Molberg et al. 2002).

For many autoimmune diseases at least one target antigen has been suggested, most often a peptide sequence found in proteins produced by tissues of the target organ. For Multiple Sclerosis (MS), the autoimmune disease that is the focus of this invention, antigens of the central nervous system (CNS) are the obvious targets of immunologic attack. For MS, the candidate antigens have been defined in vitro by showing humoral or lymphocyte reactivity to CNS-derived antigens at a level not found in normal controls. For example, the T-cell proliferative response to peptides of proteolipid protein (PLP) are significantly increased in MS patients compared to healthy subjects and this increase is also associated with the increased frequency of MS in females (Greer, Csurhes et al. 2004). The response of CD4⁺T cells to peptides of myelin basic protein (MBP) is also increased in MS patients, especially when the high-avidity MBP-specific T-cell population is measured (Bielekova, Sung et al. 2004). In addition to these well studied CNS antigens, other candidate proteins exist including myelin oligodendrocyte glycoprotein (MOG) (Mantegazza, Cristaldini et al. 2004) as well as newly described CNS autoantigens; e.g., myelin-associated oligodendrocytic basic protein (MOBP) (Holz, Bielekova et al. 2000; de Rosbo, Kaye et al. 2004).

II. Experimental Allergic Encephalomyelitis

The existence of the immune responses to CNS antigens as seen in some MS patients has been used to suggest that MS is the result of an autoimmune attack wherein the target antigen derived from the CNS. Because definitive studies can not be conducted in humans, different animal models have been developed to study the autoimmune response to CNS antigens. Most of the current studies use inbred rats or mice and the autoimmune response is initiated typically by injection of a CNS-derived neuroantigen. In order to induce clinically-apparent disease, the neuroantigen is emulsified in adjuvant (commonly complete Freund's adjuvant (CFA)). Typically, following this immunization, the experimental animal develops an ascending paralysis that initially presents as tail flaccidity and progresses to complete hindquarter paralysis. In some models, the disease continues to complete paralysis of the animal before recovery occurs. Depending on the animal used for the study (strain, species) the disease that develops is acute, recovery is complete and the animal experiences only one disease episode. In other strains, the initial disease and recovery is followed by disease relapse. These relapsing disease models are favored by many investigators because of the similar clinical condition in MS. In all cases, the disease that develops following immunization with neuroantigens is referred to as experimental allergic encephalomyelitis (EAE).

It is generally recognized that peptide specific CD4⁺T-cells cause initiation of the disease state. Because of this, many strategies to intervene to modify or prevent the expression of clinical EAE are directed at this lymphocyte population. These strategies have evolved with the increasing understanding of the immune response and its component parts, at the cellular as well as the molecular level. When successful in animals, the experimental approaches-such as those described in this invention—are considered promising candidates for use in the treatment of human autoimmune disease.

The response to neuroantigen in the SJL inbred mouse strain serves as an example of the extent of our understanding of EAE. Since many of the Examples provided in support of the invention study the development of EAE in the SJL mouse, a detailed description of the response to neuroantigen in this model is relevant. The first neuroantigen used to induce EAE in the SJL mouse strain was MBP (Fritz, Chou et al. 1983). This inbred mouse was also used to demonstrate that T cell lines with MBP specificity caused the development of clinical disease when transferred into naive recipients (Bourdette, Vandenbark et al. 1989). Subsequently, it was found that a peptide of PLP (aa 139-151) was a better encephalitogen (as compared with MBP) for the induction of EAE in SJL mice (Tuohy, Lu et al. 1989). It was also found that T-ceH lines specific to PLP caused the adoptive transfer of relapsing EAE (rEAE) following transfer into naive recipients (Zamvil, Nelson et al. 1985).

Thus, the SJL mouse strain is considered a standard model for MS due to its response to encephalitogens, the demonstrated T cell component and the relapsing nature of the disease that developed following active immunization as well as following adoptive transfer with peptide-specific T cell lines. This mouse strain has also been used to suggest the relevance of the phenomenon of epitope spreading in the course of a relapsing autoimmune disease (Yu, Johnson et al. 1996). In this study, SJL mice were injected with PLP₍₁₃₉₋₁₅₁₎, an encephalitogenic peptide that induces rEAE in this mouse strain. At intervals following immunization, spleen cells from animals were assessed for their response to this immunizing peptide and a set of overlapping peptides of the complete PLP sequence. Additional peptides from other known encephalitogenic neuroantigens were also included in the analysis. This study demonstrates a predictable spread of the T cell response to other PLP peptides and other encephalitogenic peptides. Presumably, the appearance of the T-cell populations that responded over time to these additional neuroantigens is not an epiphenomenon, but explains the relapsing nature of this disease that is initiated by the response to a single peptide. Support for the association of epitope spreading with clinical disease development is found in the fact that the following peptides are pathogenic and induce T cell responses in the following order PLP₍₁₃₉₋₁₅₁₎>PLP₍₁₇₈₋₁₉₁₎>MBP₍₈₄₋₁₀₄₎.

III. Immunotherapeutic Strategies for EAE Intervention

The SJL mouse model of EAE and other animal models have been used for the design of treatment strategies for human autoimmune disease. Many of these strategies take into account the need to be effective at the time of initial disease onset or when initiated prior to relapse. For the purposes of example, and because of the relevance to this proposal, the following discussion of immunotherapeutic strategies will be limited to those that are neuroantigen based.

Experimental models of EAE offer many advantages for the development of immunotherapeutic strategies, including the knowledge of the peptide used to induce the disease. Success in these established model systems with antigen-based strategies for inhibition of disease is important since candidate antigens exist for many human autoimmune diseases (Steinman 2004), and rational treatment alternatives do not exist (Pender and Wolfe 2002). Two of the strategies that are currently used for treatment of experimental autoimmune disease involve either: 1) monomers of the peptide covalently associated with a MHC class II molecule or; 2) linear multimers of the encephalitogenic peptide. Both reagents are made by recombinant technology and both have been tested in many systems including the SJL mouse model.

The first reagent is based on our understanding of the interaction of the TCR with its antigenic epitope. For CD4⁺T cells, the recognition of peptide antigen occurs only when the peptide is associated with the antigen-binding groove formed by the MHC class II molecule. The initial stimulation of an antigen-specific T cell via its TCR requires such MHC class II antigen presentation as well as many additional costimulatory elements (Fujii, Liu et al. 2004). Thus, the antigen-binding groove of the MHC class II molecule is a key component of the induction phase of CD4⁺T-cell immunity. Typically, MHC class II presentation of peptide takes place at the surface of an MHC class II⁺ antigen-presenting cell, characteristically a dendritic cell. The immunotherapeutic approach used by these investigators was to produce a recombinant MHC class II molecule comprised of a portion of the alpha and beta chains of the MHC class II molecule and to covalently associate an encephalitogenic peptide to the binding groove formed by the alpha and beta chains. This resulted in a monovalent ligand that the authors refer to as a recombinant TCR ligand (RTL). As used in the SJL model the RTL was comprised of relevant portions of the alpha and beta chains of the MHC class II I-A^{s} haplotype covalently associated with the PLP₍₁₃₉₋₁₅₁₎ peptide. The authors induced disease in SJL mice by immunization with monomeric PLP₍₁₃₉₋₁₅₁₎ peptide in CFA and when clinical signs of disease were evident, began treating mice with the RTL₍₁₃₉₋₁₅₁₎ peptide. The treatment consisted of eight daily injections of the specific RTL. This treatment ameliorated the clinical signs of disease and appeared to prevent relapses. Unfortunately, due to the complexity of the RTL, it is also an immunogen. Thus, mice were also treated with antihistamines as part of this treatment protocol (Huan, Subramanian et al. 2004). Nevertheless, this RTL did inhibit the full expression of clinical disease and did prevent relapses from occurring in the SJL model. This latter result may not be consistent with the concept of epitope spreading as a direct correlate of relapsing disease. Since the RTL₍₁₃₉₋₁₅₁₎ peptide was administered only after clinical disease was established, the target organ damage and presumed neuroantigen release would have occurred. This study indicates that the RTL treatment appears to cause a shift to the production of cytokines associated with regulation of pathogenic T cell expression, a finding that may contribute to the prevention of relapses following treatment with the RTL₍₁₃₉₋₁₅₁₎. This study also clearly demonstrates the antigen specific nature of the RTL₍₁₃₉₋₁₅₁₎ treatment.

The second approach to the antigen-specific modulation of autoimmune disease uses linear repeats of an encephalitogenic peptide. For use in the SJL mouse multimers of the PLP₍₁₃₉₋₁₅₁₎ peptide were produced as linear repeats of 4, 8, 12, 16 and 24 PLP peptide repeats with each peptide separated by a 13 aa spacer. All of these multimers were produced using recombinant methods developed previously by these investigators (Rotzschke, Falk et al. 1997). The various PLP peptide multimers were tested in vitro and a 16-mer of the PLP peptide repeat was selected for subsequent in vivo studies. In vivo it was found that this 16-mer was more encephalitogenic than the monomeric PLP peptide when injected into SJL mice. In the absence of CFA, neither the monomer nor multimer was encephalitogenic. The 16-mer was then used as a saline solution and was found to completely inhibit the development of EAE was administered to SJL mice prior to (treatment day −7 and −4) or after (treatment day +3, +7) immunization of the mice with CFA containing the PLP₍₁₃₉₋₁₅₁₎ peptide. This inhibition appeared to be specific in that mice immunized with the encephalitogenic PLP₍₁₇₈₋₁₉₁₎ peptide develop EAE even though treated with the PLP₍₁₃₉₋₁₅₁₎ peptide 16-mer (Falk, Rotzschke et al. 2000). Of interest is the observation that the PLP₍₁₃₉₋₁₅₁₎ peptide 16-mer inhibited the development of EAE induced by the injection of spinal cord homogenate emulsified in adjuvant. This latter observation complicates the analysis in that many of the different neuroantigens contained within the spinal cord homogenate have been shown to be encephalitogenic. The response elicited by these additional encephalitogens contained within the spinal cord homogenate should have resulted in the development of EAE. Inhibition of the response to the major PLP encephalitogenic epitope may influence subsequent responses to other epitopes.

The studies using RTL and the studies using linear peptide multimers demonstrate that presentation of the relevant epitope to the responding immune system can result in the deviation of the clinical manifestation of an autoimmune disease. There are additional studies showing the efficacy of these reagents in other animal models of autoimmune disease (Burrows, Bebo et al. 1998; Burrows, Adlard et al. 2000; Falk, Rotzschke et al. 2000; Stienekemeier, Falk et al. 2001; Vandenbark, Rich et al. 2003). The two approaches described above are two examples of antigen specific therapy for autoimmune disease. Many different strategies have been considered for antigen specific inhibition of disease, (reviewed in (Harrison and Hafler 2000)), and have included direct chemical conjugation of the encephalitogen to spleen cells which are then injected into experimental animals to modify disease expression (Vandenbark, Celnik et al. 1995; Vanderlugt, Neville et al. 2000).

IV. Interventional Strategies of the Invention

The present invention is the result of the unexpected discovery that peptide multimers, chemically synthesized as octamers of the encephalitogenic peptide, successfully induce antigen-specific alteration in autoimmune disease, as described in more detail in the Examples. The utility and mechanism of action of these octamers are presented below.

For obvious practical and moral reasons, initial work in humans to determine the efficacy of experimental compositions or methods with regard to many diseases is infeasible. Thus, during early development of any drug it is standard procedure to employ appropriate animal models for reasons of safety and expense. The success of implementing laboratory animal models is predicated on the understanding that immunodominant epitopes are frequently active in different host species. Accordingly, the pathologic target of the immune response that leads to an autoimmune disease in one species, for example a rodent, will generally share common features when evaluated in a different species such as in humans. Only after the appropriate animal models are sufficiently developed will clinical trials in humans be carried out to further demonstrate the safety and efficacy of the present invention. Accordingly, for purposes of explanation only and not for purposes of limitation, the present invention will be primarily demonstrated in the exemplary context of mice as the mammalian host. Those skilled in the art will appreciate that the present invention may be practiced with other mammalian hosts including humans.

In summarizing experiments conducted in support of the invention, it was observed that chemically synthesized multiple antigen peptides (MAPs), as peptide octamers, are highly effective reagents for the antigen-specific inhibition of EAE in both acute and relapsing disease models of this experimentally-induced autoimmune disease. We have found that octamers of the 139-151 amino acid peptide of proteolipid protein (139MAP) completely prevents or significantly inhibits the development of both actively-induced as well as adoptively transferred forms of this disease. In addition, 139MAP causes a rapid decrease in clinical signs of EAE that have developed due to adoptive transfer of encephalitogenic peptide-specific T-cell lines or following immunization with the encephalitogenic 139-151 aa peptide of PLP in complete Freunds adjuvant (CFA). This is an antigen-specific inhibition of the ongoing disease and 139MAP treatment is effective in preventing or significantly reducing the relapsing disease that develops in the SJL mouse model. Additional studies suggest that 139MAP treatment alters lymphoid traffic to the autoimmune target organ and that for a time the antigen specific lymphocytes remain in the secondary lymphoid organ. Further observations suggest that these cells may lose their potential for subsequent response to antigen, an observation that may be due to the development of anergy (Mannie, Rendall et al. 1996; Chou, Robey et al. 1998) in this antigen-specific T-cell population. This time dependent loss of antigen responsiveness may also be due to a delayed-deletion of the antigen-specific T cell following the in vivo response to 139MAP (Hawiger, Inaba et al. 2001).

The effectiveness of this type of antigen multimer provides obvious utility in the treatment of a variety of autoimmune diseases. It is recognized that MAPs are used in conjunction with adjuvants typically to enhance an immune response. Part of their effectiveness in the rat and mouse EAE models used in the experiments conducted in support of the present invention may reflect a greater stability of MAPs in vivo compared to other antigen specific reagents (Bracci, Falciani et al. 2003). Specific applications of the invention to four autoimmune indications are described in the following section (V).

V. Therapeutic Treatment of Autoimmune Diseases Using the Compositions and Methods of the Invention

As described above, an objective of the present invention is to provide a therapeutic approach to the treatment of multiple sclerosis (MS). Other autoimmune diseases can be treated using the compositions and methods of the invention. Exemplary human autoimmune diseases are described below, including MS, that are expected to benefit from therapeutic intervention using MAP octamers that incorporate known immunodorninant epitopes for the specific disease.

Whatever form of MAP octamer selected, the compositions of the present invention may be formulated to provide desired stability and facilitate the selected form of administration. For example, the compositions may be administered using all the conventional routes including, but not limited to, oral, vaginal, aural, nasal, pulmonary, intravenous, intracranial, intraperitoneal, subcutaneous, or intramuscular administration (see also Definitions supra). Within other embodiments of the invention, the compositions described herein may be administered as part of a sustained release implant. Within yet other embodiments, compositions of the present invention may be formulated as a lyophilized or spray dried formulation, utilizing appropriate excipients which provide stability.

A further aspect of the invention comprises a method for treating an autoimmune disorder comprising administering to a patient a therapeutically effective amount of a pharmaceutical composition comprising a multiple antigen peptide in combination with a physiologically acceptable carrier or diluent wherein said antigen-specific immunosuppressive agent comprises at least four and at most twelve antigen peptides. As previously alluded to, the multiple antigen peptide agent will preferably be in the form of multiple synthetic polypeptides linked to a central core. The method may be used to treat immune disorders comprising autoimmune disorders, allergic responses and transplant rejection, and is particularly usefuil in treating autoimmune disorders selected from the group consisting of multiple sclerosis, Type I diabetes, rheumatoid arthritis, psoriasis, pemphigus vulgaris and Celiac disease.

Pharmaceutical compositions of the present invention may be administered in a manner appropriate to the disease to be treated (or prevented). The quantity and frequency of administration will be determined by such factors as the condition of the patient, and the type and severity of the patient's disease. Within particularly preferred embodiments of the invention, the pharmaceutical compositions described herein may be administered at a dosage ranging from 1 mg to 50 mg/kg, although appropriate dosages may be determined by clinical trials. Those skilled in the art will appreciate that patients may be monitored for therapeutic effectiveness by MRI for signs of clinical exacerbation.

A. Multiple Sclerosis

MS is an immune-mediated inflammatory disease that causes multiple lesions in the central nervous system (CNS) such as in the brain and spinal cord to induce various neurological symptoms (blurred vision, dyskinesia, decreased sensation, abnormal sensation, pain, loss of balance/tremor, urinary problems, sexual dysfunction, fatigue, cognitive/emotional disorder, etc.). MS has not been etiologically explained, but it is considered an autoimmune disease in which the immune system accidentally attacks its own tissue. This disease is thought to be caused by a process in which T-cells and macrophages infiltrate into the white matter to incorrectly recognize the body's “own myelin” covering the axons of neurons in the brain or spinal cord as a foreign enemy and attack it, resulting in inflammation of myelin and then demyelination (destruction of myelin).

Current MS therapy falls into the following three categories: inflammation relief during the acute phase, prevention of relapse or progression, and symptom relief During the acute phase, adrenocortical steroids are used to reduce inflammation in demyelinated regions. Interferon and immunosuppressants are thought to be effective for preventing relapse or progression. In addition, transforming growth factor beta (TGF-0), which possesses suppressive activity against T-cell proliferation, has been reported to inhibit experimental allergic encephalomyelitis (EAE).

Although immunologically-based treatments have been extensively investigated based on the pathogenic mechanisms of MS, effective and satisfactory therapeutic methods are not well established. Many MS patients alternate between relapse (inflammation in the brain or spinal cord) and remission (recovery from relapse), but are difficult to completely cure. It is therefore desirable to develop novel and effective therapeutic agents. One approach has been the administration of altered peptide ligand (APL) peptides with amino-acid substitutions in T-cell receptor contact positions. This strategy may block T-cell responses by acting as partial agonists, TCR antagonists, or by inducing regulatory T-cell populations that mediate bystander suppression. However, use of an APL of myelin basic protein, MBP₍₈₃₋₉₉₎, resulted in worsening disease and cessation of a human clinical trial (Bielekova, Goodwin et al. 2000).

The MBP₍₈₃₋₉₉₎ (SEQ ID NO: 1) peptide is known to be immunodominant in humans in the context of MS-associated human leukocyte antigen DR molecules (WO9616086). MBP₍₈₃₋₉₉₎ is therefore predicted to offer therapeutic efficacy when administered to MS patients in the form of an MAP octamer. Use of the peptide MBP₍₈₃₋₉₉₎, in a MAP octamer offers therapeutic advantages as described for analogous peptides in the Examples without the undesirable side effects observed when the peptide is used as a monomer, as described (Bielekova, Goodwin et al. 2000).

Immunodominant peptides have been used in mice in efforts to suppress the development of EAE, as described above. Anaphylactic reactions have been reported in studies in which mice that had been immunized with PLP-derived or myelin oligodendrocyte glycoprotein (MOGP)-derived peptides in an attempt to suppress EAE development. Furthermore, hypersensitivity reactions have caused the premature discontinuation of MBP-derived peptide therapy in human subjects with MS (Bielekova, Goodwin et al. 2000; Kappos, Comi et al. 2000). Therefore, the use of these peptides, using the compositions and methods of the present invention, in the form of MAP octamers, potentially avoids the unintended side effects described in the use of these previous approaches.

B. Type 1A Diabetes

Type 1 diabetes is an autoimmune disease resulting from the selective destruction of insulin-producing β cells in the pancreas by autoreactive T cells. Overt type 1 diabetes (also known as insulin-dependent diabetes mellitus or early onset diabetes) is often preceded by the appearance of insulin autoantibodies. Prophylactic administration of insulin to diabetes-prone rats, nonobese diabetic (NOD) mice, and human subjects results in protection from diabetes. This suggests that an immune response to insulin is involved in the process of beta cell destruction. Islet-infiltrating cells isolated from NOD mice are enriched for insulin-specific T cells and insulin-specific T cell clones are capable of adoptive transfer of diabetes. Epitopes present on residues 9-23 of the insulin B chain (B₍₉₋₂₃₎) appear to be dominant in this spontaneous response (Daniel and Wegmann 1996). On the basis of these observations, the impact of subcutaneous or intranasal administration of B₍₉₋₂₃₎ on the incidence of diabetes in NOD mice was examined and resulted in a marked delay in the onset and a decrease in the incidence of diabetes. This protective effect is associated with a reduced T-cell proliferative response to B₍₉₋₂₃₎ in treated mice (Daniel and Wegmann 1996). However, peptide therapy using B(₉2₃) or a similar peptide B₍₁₃₋₂₃₎ is compromised by the development of anaphylaxis in these mice (Liu, Moriyama et al. 2002). The same research group has attempted to modify the B₍₉₋₂₃₎ peptide to decrease anaphylaxis and have achieved a degree of success in the NOD mouse model (Liu, Moriyama et al. 2004).

The autoantigen glutamic acid decarboxylase 65 (GAD 65) is thought to be an important target antigen in type 1 diabetes and plays a key role in the pathogenesis of the disease (Ellis and Atkinson 1996; Chao and McDevitt 1997). Immunogenic epitopes of GAD 65 have been identified using the non-obese diabetic mouse (NOD) model (Chao and McDevitt 1997). It is also known that certain of these immunogenic peptides can induce antigen-specific T regulatory cells that protect NOD mice from developing disease (You, Chen et al. 2004). Two GAD 65 peptides, GAD₍₂₀₆₋₂₂₀₎ and GAD₍₂₂₁₋₂₃₅₎ (SEQ ID NOS:9 and 10, respectively), are of particular interest, and combined with the present invention, offer a therapeutic approach to preventing type 1 diabetes.

The use of the B(9-23) peptide (SEQ ID NO:2) or the GAD(206-220) or GAD(221-235) peptides (SEQ ID NOS:9 and 10, respectively) using the methods and compositions of the present invention, in the form of MAP octamers, offer an immunomodulating therapeutic strategy to prevent type 1 diabetes and avoids the anaphylactic response observed using conventional approaches.

Recent evidence suggests that insulin is an essential autoimmune target in the initiation of diabetes (Kent, Chen et al. 2005; Nakayama, Abiru et al. 2005; von Herrath 2005). Accordingly, use of the I₍₃₃₋₄₇₎ peptide (SEQ ID NO:11) offers an additional immunomodulating therapeutic strategy to prevent type 1 diabetes.

C. Pemphilus Vulgaris

Pemphigus vulgaris (PV) is an autoimmune blistering disease of the skin and mucous membranes and is histologically characterized by intraepidermal blister formation and immunopathologically characterized by autoantibody against the keratinocyte cell surface (Amagai, Klaus-Kovtun et al. 1991). Clinically, patients with pemphigus vulgaris exhibit extensive flaccid blister and erosion. Pemphigus vulgaris can be lethal since failing to conduct an appropriate therapy results in the induction of the leakage of body fluid or secondary bacterial infection caused by the focus developed at a wide range in the skin. The prognosis of pemphigus is being improved by systemic administration of corticosteroid and immunosuppression therapy, however, its mortality rate remains very high because of complications associated with this therapy.

PV is caused by autoantibodies against the extracellular domain of desmoglein 3 (Dsg3) (Amagai, Klaus-Kovtun et al. 1991). Dsg3-reactive T helper 1 and 2 cells (T1 and Th2, respectively) have been identified in patients with active PV (Lin, Swartz et al. 1997; Veldman, Stauber et al. 2003). It is also known that autoaggressive T cells of PV patients recognize a limited set of Dsg3 peptides (Veldman, Gebhard et al. 2004). Three peptides (SEQ ID NOS:3-5) are preferentially recognized by a majority of autoaggressive T cells from PV patients: Dsg3₍₉₆₋₁₁₂₎; Dsg3₍₂₀₅₋₂₂₁₎; and Dsg3₍₂₅₀₋₂₆₆₎, respectively. These peptides, when used according to the methods and compositions of the present invention, offer the potential to modify the autoimmune impact of these pathogenic T cells and thus to reduce or eliminate the clinical signs of this serious autoimmune disease.

D. Celiac Disease

Celiac disease (CD), also known as gluten-sensitive enteropathy or Celiac Sprue, is an autoimmune disease of the small intestine caused by the ingestion of gluten proteins from a variety of sources including wheat, rye, and barley. Clinical symptoms of CD include fatigue, chronic diarrhea, malabsorption of nutrients, weight loss, abdominal distension, anemia, as well as a substantially enhanced risk for the development of osteoporosis and intestinal malignancies such as lymphoma and carcinoma. The disease has an incidence of approximately 1 in 200 in European populations and is believed to be significantly under diagnosed in other populations. Antibodies found in the serum of CD patients support the theory that the disease is immunological in nature and appear in almost 100% of the patients with active CD. Intestinal damage is thought to be caused by interactions between specific gliadin oligopeptides and the HLA-DQ2 or DQ8 antigen, which in turn induce proliferation of T lymphocytes in the sub-epithelial layers (Shan, Molberg et al. 2002). T helper 1 (Th1) cells and cytokines apparently play a major role in a local inflammatory process leading to villous atrophy of the small intestine. Currently, there is no effective therapy for the disease except the complete avoidance of all foods containing gluten, a difficult task given the widespread use of gluten in the modern diet. Although gluten withdrawal has transformed the prognosis for children and substantially improved it for adults, some people still die of the disease. These are mainly adults who had severe disease at the outset. A leading cause of death is lymphoreticular disease, especially intestinal lymphoma.

Efforts to identify the immunodominant epitope have pointed to a peptide of alpha-gliadin that includes amino acids 56 to 75 (αG₍₅₆₋₇₅₎) (Ellis, Pollock et al. 2003; Fraser, Engel et al. 2003). This peptide (SEQ ID NO:6), when used according to the methods and compositions of the present invention, offers the potential to induce an antigen-specific alteration in this serious autoimmune disease.

Materials and Methods

I. Preparation of Multimeric Peptides of the Invention

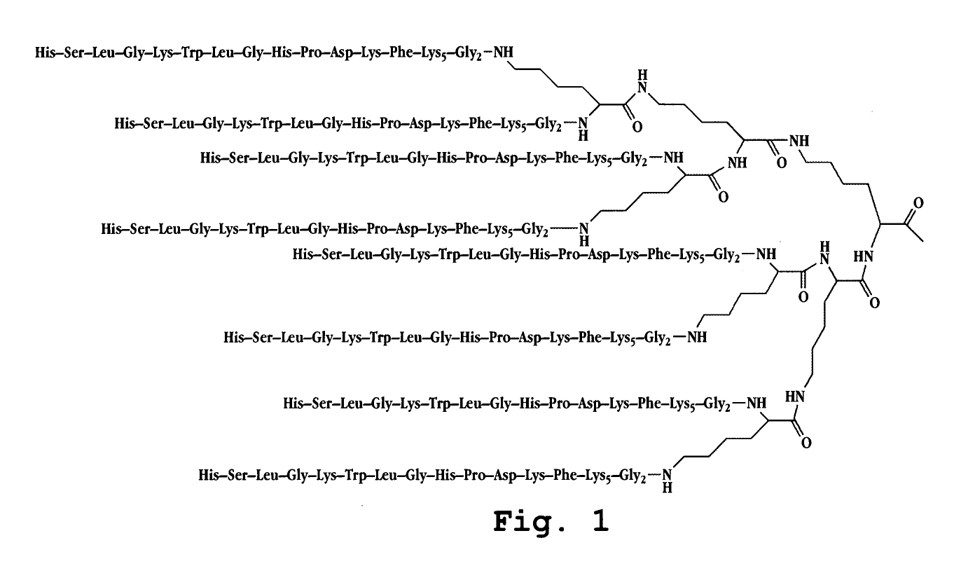

Solid phase synthesis procedures that are commonly employed for the preparation of multiple antigen peptides (MAPs) have been described (Lu, Clavijo et al. 1991; Tam 1996) and were used to prepare the following two MAPs: 1) chemically synthesized octamers of the 72-85 amino acid peptide (72-85 aa) of guinea pig myelin basic protein (gpMBP, SEQ ID NO:7) and; 2) chemically synthesized octamers of the 139-151 amino acid peptide of proteolipid protein (139-151 PLP MAP or 139MAP, SEQ ID NO:8). The starting polymer matrix is copoly(styrene-1% DVB) with a central core molecule, Fmoc₈-Lys₄-Lys₂-Lys-betaAla, linked to the Wang resin (Novabiochem, catalog number 05-24-0150). Eight peptides are covalently linked to the central core by displacement of the eight Fmoc residues resulting in an octameric multiple antigen peptide molecule as shown in FIG. 1 for 139MAP. In the case of the MAP139 molecule shown in FIG. 1, a seven amino acid spacer, NH₃—KKKKKGG-COOH, is used to separate the central core from the antigenic peptide. This spacer is not a necessary component in practicing the invention. The composition and sequence of the spacer shown in FIG. 1 is exemplary and alternative sequences may be used. For example, a spacer consisting of NH₃-GG-COOH may be used in place of NH₃—KKKKKGG-COOH with equivalent results.

II. Clinical Scoring of EAE

Animals are graded as grade 1 when the first sign of disease is evident which is a flaccid tail. As the clinical signs of disease become more severe, the disease score is increased to a maximum of 7, a score representing complete paralysis (Huan, Subramanian et al. 2004).

### EXAMPLES

### Example 1

### Prevention of EAE in Lewis Rats Treated With 72-85gpMBP MAP Octamers

Experimental autoimmune encephalomyelitis (EAE) in the rat is an acute paralytic disease from which most animals spontaneously recover. The disease can be induced in susceptible inbred Lewis and DA rats with myelin basic protein (MBP), or encephalitogenic MBP peptides administered in complete Freund's adjuvant (CFA). The disease can be adoptively transferred to syngeneic recipients with primed T cells that have been reactivated in vitro with antigen. Studies of EAE in susceptible rats have provided many important insights into the interactions of T cells and accessory cells that culminate in the induction of the autoimmune response.

The influence of a saline solution of the 72-85gpMBP octamer on the course of EAE in Lewis rats previously injected with gpMBP emulsified in CFA was investigated. We injected 10 or 50 micrograms of the 72-85 gpMBP octamer into Lewis rats 4 and 8 days after they had been injected with gpMBP-CFA. The group of Lewis rats that received the 50 micrograms dose did not develop any signs of clinical disease. The group of rats that received the 10 micrograms dose developed less severe disease compared to the control group. Rats that received an equivalent amount of the 72-85 amino acid gpMBP peptide monomer at day 4 and day 8 developed clinical signs of disease. No diminution of the signs of clinical disease was seen in the 10 micrograms treatment group, while a modest diminution of the clinical signs of EAE was seen in the rats receiving 50 micrograms of the peptide monomer.

These initial results demonstrated the ability of octamers to retain encephalitogenic activity while causing a response deviation when administered in nonencephalitogenic form (e.g., in saline). Although the Lewis rat is a well-studied model of EAE, the clinical disease is monophasic and acute with peak disease typically evident for at most 2 days and the disease course completed in 4-6 days. Because of this limitation, the SJL mouse was used in additional experiments described below. It has been shown that immunization of the SJL mouse strain with encephalitogenic peptide in CFA causes the onset of severe clinical EAE approximately 2 weeks following immunization (Whitham, Jones et al. 1991; Tuohy, Thomas et al. 1995). The SJL mouse subsequently recovers from this initial disease episode and then relapses and exhibits another round of clinical disease. Many animals have an additional relapse while others develop a chronic form of the disease. We also chose to work in the SJL mouse strain since others have used this strain to demonstrate the activity of their immunotherapeutic reagents (Falk, Rotzschke et al. 2000; Huan, Subramanian et al. 2004). This allowed a comparison of their strategies with the experiments in support of the present invention.

### Example 2

### Prevention of EAE in SJL Mice Treated With PLP₍₁₃₉₋₁₅₁₎ MAP Octamers

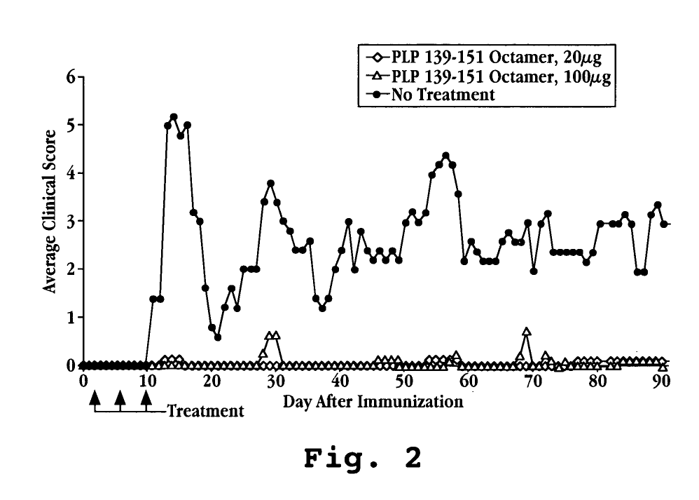

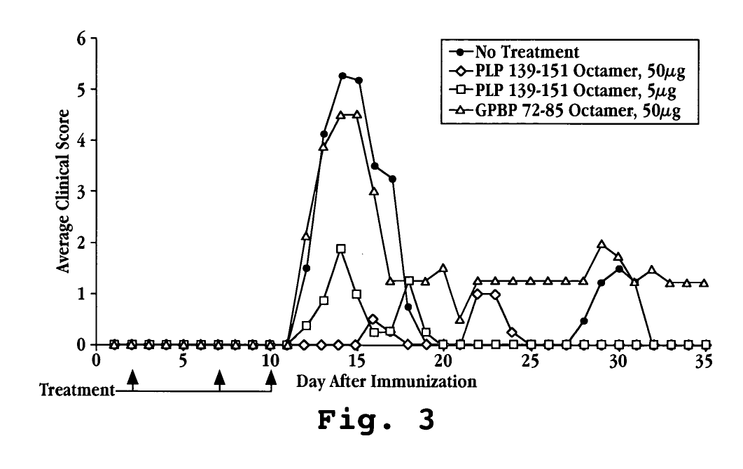

The following experiments in support of the invention demonstrate the immunoregulatory properties of MAPs. For all of these studies, MAP octamers of the 139-151aa peptide of proteolipid protein (PLP) were used and prepared by solid phase peptide synthesis as described above in the Materials and Methods. Each PLP₍₁₃₉₋₁₅₁₎ MAP octamer is comprised of eight polypeptide chains of the 139-151 aa of PLP each covalently linked to a central core as shown in FIG. 1. The general structure of the MAP (FIG. 1) is therefore dissimilar to the linear array typical of the peptide multimers used by others in the prior art (Falk, Rotzschke et al. 2000; Falk, Rotzschke et al. 2000; Stienekemeier, Falk et al. 2001). MAPs are also referred to as dendrimers to emphasize the branched nature of their structure (Bracci, Falciani et al. 2003). Initial studies with the 139-151 PLP MAP octamers (139MAP) used SJL mice that were first immunized with the encephalitogenic 139-151 PLP peptide in CFA (139CFA) and subsequently injected with saline solutions of 139MAP. FIG. 2 shows that saline injections of 139MAP prevented the development of clinical signs of EAE when administered to mice that been injected with 139CFA. For this initial study, mice were treated by intraperitoneal or subcutaneous injection with 139MAP on days 2, 7 and 10 following 139CFA injection. Virtually all signs of disease were prevented by a dose of 100 micrograms as well as 20 micrograms of the 139MAP. FIG. 3 extends this observation and shows the antigen-specific inhibition of the MAP influence in that the 72-85 MAPs, that were effective an altering the course of EAE in the Lewis rat had no effect of the development of EAE disease in SJL mice immunized with 139CFA. It is evident that three injections of 5 micrograms of 139MAP cause a considerable diminution of EAE relative to the mice that received no MAP injection or the group that received the 72-85 gpMBP peptide octamers.

### Example 3

### Initial Clinical Signs of EAE are Reversible Upon Treatment With MAP139

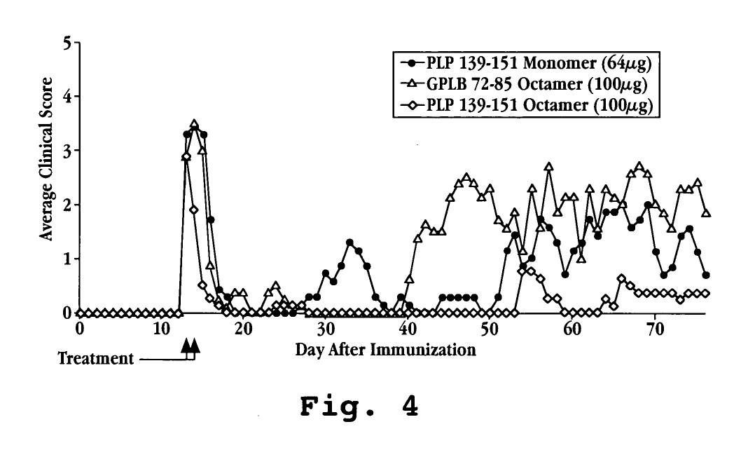

Inhibition of the development of EAE with MAP 139 as described in Example 2 provided a rationale for a series of experiments that examined the influence of 139MAP when administered to SJL mice showing initial signs of clinical EAE disease that developed following immunization with 139CFA. Treatment with 139MAP, initiated after the development of actively induced EAE, had a profound and immediate influence on the subsequent course of disease in 139CFA immunized mice. Shown in FIG. 4 are the results of experiments in which mice were treated with the 139MAP when clinical disease was evident, typically with disease grades 1-3. The 139MAP-treated animals were given 100 microgram doses of 139MAP on two consecutive days. Some animals in the treatment group showed mild relapses with the majority of the treated animals exhibiting no additional disease episodes. The studies in FIG. 4 compare the ability of the PLP₍₁₃₉₋₁₅₁₎ monomer, with the 72-85gpMBP MAP and the 139MAP, to inhibit relapsing EAE (rEAE). All reagents were administered on two consecutive days after clinical disease was evident. It should be noted that we used an equal dose of the PLP monomer and the 139MAP based on the content of monomer sequence that contributes to the final 139MAP product. FIG. 4 also shows that the 139MAP inhibited the relapse component of this disease. The 139-151 monomer had no influence on the course of disease or on the relapses that develop following the initial disease episode. We did not see exacerbation of the disease following the monomer treatment as has been found by others (Falk, Rotzschke et al. 2000). In this study, we chose the 100 microgram dose in order to use the monomer at a dose that has reported to have some influence on disease development. It is anticipated that lower doses of 139MAP will have a more significant inhibitory effect than the doses employed in this study.

### Example 4

### Inhibition of EAE by 139MAP is Antigen-Specific

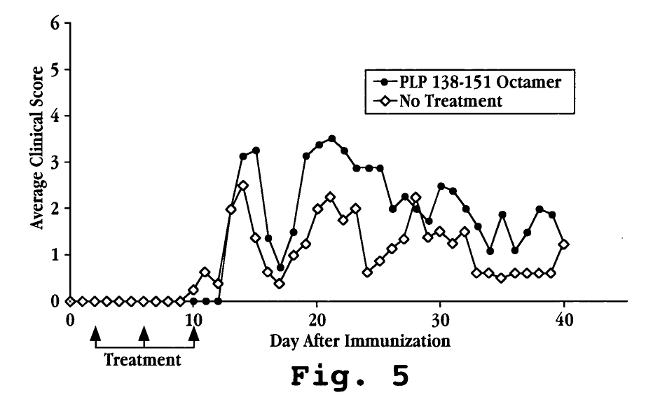

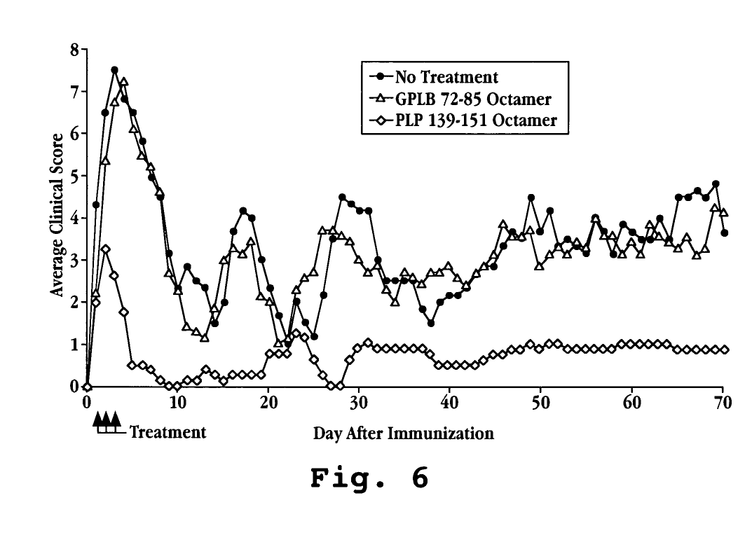

The relapsing disease that develops in SJL mice is thought to occur due to the immune response that develops against additional encephalitogenic determinants found in PLP and MBP (Cross, Tuohy et al. 1993). In order to demonstrate the epitope specificity of 139MAP and to provide insight into the long-term inhibition of relapses seen following 139MAP treatment, SJL mice were immunized with a second encephalitogenic peptide of PLP, the 178-191 aa peptide (PLP₍₁₇₈₋₁₉₁₎). Immunization of SJL mice with the PLP₍₁₇₈₋₁₉₁₎ peptide in CFA caused the development of clinically apparent disease 12-14 days following immunization. FIG. 5 shows the results of one experiment in which groups of animals were treated with 139MAP on days 2, 7 and 10 following immunization with the 178-191 PLP peptide in CFA. In this initial study, we saw no significant inhibition of disease development as mediated by 139MAP. This is in sharp contrast to the data shown in FIG. 2 and suggests that the 139MAP inhibits the development and expression of EAE in an antigen specific manner. FIG. 6 represents a second experiment (4 animals per group) in which mice treated with 139MAP after they developed initial signs of EAE due to immunization with the PLP₍₁₇₈₋₁₉₁₎ peptide. In this group the 139MAP inhibits the development of relapses. This result is consistent with our understanding of the development of disease in the SJL mouse model and is in keeping with the observation that the PLP₍₁₃₉-l1l) epitope is the pathogenic target during the first relapse in studies where the initial disease episode was induced by other encephalitogenic epitopes (Vanderlugt, Neville et al. 2000).

### Example 5

### EAE Induced by Adoptive Transfer of T-Cells Can be Blocked by 139MAP

In addition to the use of the actively induced model of EAE to establish the immunoregulatory features of 139MAP, studies that have used encephalitogenic T-cell lines have provided additional insight into the in vivo influence of 139MAP on disease development. The T-cell lines employed for these studies were derived from SJL mice previously immunized with 139CFA. The T-cell lines are used for adoptive transfer studies following the third round of antigen-stimulation in vitro. The source of antigen presenting cells is irradiated syngeneic thymus cells. This is a procedure that is routinely used by other investigators and is known by those of skill in the art (Bourdette, Vandenbark et al. 1989).

A representative T-cell line that proliferated in response to the PLP₍₁₃₉₋₁₅₁₎ monomer and the 139MAP octamer was used to adoptively transfer clinical disease to naive SJL mice. As shown in FIG. 6, transfer of the PLP₍₁₃₉₋₁₅₁₎ peptide specific T-cell lines caused the development of disease in all cell recipients. The animals receiving no additional treatment as well as animals receiving the control 72-85 octamer developed a relapsing episode of clinically severe EAE. We treated some of the cell recipients with 50 micrograms 139MAP on three consecutive days with treatment initiated after the development of clinical signs of EAE. As shown in FIG. 6, the treatment with 139MAP inhibited the continued development of the first episode of disease and this group of animals subsequently developed no or very modest signs of additional episodes of EAE. It was also observed that the development of adoptively transferred EAE and subsequent relapses can be completely inhibited if 139MAP is administered prior to the onset of disease and when animals are treated at the first indication of disease (tail flaccidity) (data not shown). This is very clear in vivo data showing the 139MAP-associated inhibition of rEAE in the SJL mouse model. The 139MAP reagent inhibits the progression of clinically apparent disease as well as the onset and the continued development of EAE in both the actively-induced as well as the adoptively-transferred forms of EAE.

It is claimed:

1. A composition for use in treating an autoimmune disorder, comprising
(a) an immunomodulating compound composed of a central amino acid core having a plurality of chemical attachment groups, and a plurality of antigenic peptides which are (i) associated with an auto-immune disorder and (ii) attached to said groups with the same N-terminus to C-terminus orientation, and
(b) a pharmaceutically acceptable carrier.

2. The composition of claim 1, wherein each of said antigenic peptides is attached to said amino acid core through a spacer containing a linear chain of at least about 5 atoms in length.

3. The composition of claim 1, wherein said central amino acid core is a branched polylysine core having between 4-16 attachment sites, and the compound includes between 4-16 antigenic peptides.

4. The composition of claim 3, wherein said core is composed of a polylysine octamer, and said compound includes up to eight antigenic peptides, each coupled to said octamer through the peptide's C terminus.

5. The composition of claim 3, wherein each of said antigenic peptides is attached to said polylysine core through a spacer containing a linear chain of at least about 5 atoms in length.

6. The composition of claim 5, wherein said spacer is composed of a linear chain of at least two amino acids.

7. The composition of claim 5, wherein said spacer has the sequence identified by SEQ ID NO:12.

8. The composition of claim 3, wherein said antigenic peptide is selected from the group consisting of SEQ ID NOS:1-11.

9. The composition of claim 1, wherein said carrier is buffered physiological saline.

10. The composition of claim 1, wherein central amino acid core is a branched polylysine octamer having eight amine attachment sites, and said antigenic peptides are attached at the C termini to said sites, each through a spacer composed of a linear chain of at least five amino acids, and the antigenic peptides are selected from the group consisting of SEQ ID NOS:1-11.

11. The composition of claim 10, wherein said compound includes eight antigenic peptides having selected from the group consisting of SEQ ID NOS:1-11, and each peptide is linked to a polylysine amine attachment group through a spacer having the sequence identified by SEQ ID NO:12.

12. A method of treating an autoimmune disorder in a mammalian subject in need of such treatment comprising
administering to said subject, in a pharmaceutically effective amount, a composition containing (a) an immunomodulating compound composed of a central amino acid core having a plurality of chemical attachment groups, and a plurality of antigenic peptides which are (i) associated with an auto-immune disorder and (ii) attached to said groups with the same N-terminus to C-terminus orientation, and (b) a pharmaceutically acceptable carrier.

13. The method of claim 12, wherein said administering includes administering a compound whose central amino acid core is a branched polylysine core having between 4-16 attachment sites, and which has between 4-16 antigenic peptides, each attached at their C termini through a spacer containing a linear chain composed of at least five amino acids.

14. The method of claim 13, for use in treating multiple sclerosis, wherein the compound administered contains, as the antigenic peptides, a peptide selected from the group consisting of SEQ ID NOS:1, 7, or 8.

15. The method of claim 13, for use in treating early-onset diabetes, wherein the compound administered contains, as the antigenic peptides, a peptide selected from the group consisting of SEQ ID NOS:2, 9, 10, or 11.

16. The method of claim 13, for use in treating Pemphigus vulgaris, wherein the compound administered contains, as the antigenic peptides, a peptide selected from the group consisting of SEQ ID NOS:3-5.

17. The method of claim 13, for use in treating Celiac disease, wherein the compound administered contains, as the antigenic peptides, the peptide identified as SEQ ID NO:6.


SEQUENCE LISTING (12 sequences)

SEQ ID NO: 1 - protein, 17 aa, Artificial Sequence
  Synthetic Peptide
ENPVVHFFKNIVTPRTP

SEQ ID NO: 2 - protein, 15 aa, Artificial Sequence
  Synthetic Peptide
SHLVEALYLVCGERG

SEQ ID NO: 3 - protein, 17 aa, Artificial Sequence
  Synthetic Peptide
PFGIFVVDKNTGDINIT

SEQ ID NO: 4 - protein, 17 aa, Artificial Sequence
  Synthetic Peptide
GTPMFLLSRNTGEVRTL

SEQ ID NO: 5 - protein, 17 aa, Artificial Sequence
  Synthetic Peptide
QCECNIKVKDVNDNFPM

SEQ ID NO: 6 - protein, 20 aa, Artificial Sequence
  Synthetic Peptide
LQLQPFPQPQLPYPQPQLPY

SEQ ID NO: 7 - protein, 14 aa, Artificial Sequence
  Synthetic Peptide
LPQKSQRSQDENPV

SEQ ID NO: 8 - protein, 13 aa, Artificial Sequence
  Synthetic Peptide
HSLGKWLGHPDKF

SEQ ID NO: 9 - protein, 15 aa, Artificial Sequence
  Synthetic Peptide
TYEIAPVFVLLEYVT

SEQ ID NO: 10 - protein, 15 aa, Artificial Sequence
  Synthetic Peptide
LKKMREIIGWPGGSG

SEQ ID NO: 11 - protein, 15 aa, Artificial Sequence
  Synthetic Peptide
SHLVEALYLVCGERG

SEQ ID NO: 12 - protein, 7 aa, Artificial Sequence
  Synthetic Peptide
KKKKKGG